(19) United States
(12) Patent Application Publication
(10) Pub. No.: US 2009/0143623 A1
(43) Pub. Date: Jun. 4, 2009

(54) Production Process of Cyanoethyl Ether

(75) Inventors: Taiji Katsumata, Minami-Ashigara-shi (JP); Takayuki Ito, Minami-Ashigara-shi (JP); Masayuki Harada, Minami-Ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/276,524

(22) Filed: Nov. 24, 2008

(30) Foreign Application Priority Data
Nov. 29, 2007 (JP) 2007-308864
May 12, 2008 (JP) 2008-125255

(51) Int. Cl. C07C 43/04

(52) U.S. Cl. 568/589

(57) ABSTRACT

Provided is a production process of a cyanoethyl ether by reacting an alcohol and acrylonitrile in a two-phase system of a non-ether solvent/an aqueous alkali solution.

### BACKGROUND OF THE INVENTION

1. Field of the Invention

The present invention relates to a production process of a cyanoethyl ether by reacting an alcohol and acrylonitrile, more specifically, a production process of a high-purity cyanoethyl ether in a high production yield while ensuring safety in human health and environment.

2. Description of the Related Art

Cyanoethyl ethers are useful as medicaments, agricultural chemicals, and various functional materials, or intermediates therefor. These cyanoethyl ethers can usually be synthesized by the Michael addition reaction between an alcohol and acrylonitrile in the presence of a base. For example, it is known that pentaerythritol and acrylonitrile are reacted in the presence of sodium hydroxide to yield the corresponding tetracyanoethyl ether in a high yield (refer to, for example, Journal of American Chemical Society, 118(49), 12368-12375(1996) and Tetrahedron Letters, 37(8), 1237-1240(1996)). Such a reaction is however performed in a solventless manner so that it is not suited for mass production because of difficulty in control of a reaction rate or temperature. As an example of overcoming this problem, known is an example of reacting pentaerythritol and acrylonitrile in the presence of an aqueous solution of potassium hydroxide while using dioxane as a solvent and obtaining the corresponding tetracyanoethyl ether (refer to, for example, Journal of Fluorine Chemistry, 125(5), 749-754(2004), Journal of Organic Chemistry, 65(26), 9210-9213(2000), and Tetrahedron, 55(10), 2941-2956(1999)). Dioxane used as the solvent is however a substance of concern for carcinogenicity and a severe restriction is imposed on the use of it. Another problem of the above-described process is that dichloromethane is used in order to remove a coloring component formed as a by-product in the reaction, though use of it must be avoided as much as possible from the viewpoint of toxicity and environmental burden.

### SUMMARY OF THE INVENTION

An object of the invention is to provide an industrially advantageous process for producing a high-purity cyanoethyl ether from an alcohol and acrylonitrile in a high production yield while ensuring safety in human health and environment.

As a result of an extensive investigation with a view to achieving the above-described object, the present inventors have found the processes described below and completed the invention. In the invention, there are thus provided the following means for resolution.

(1) A process for producing a cyanoethyl ether, comprising:

reacting an alcohol and acrylonitrile in a two-phase system of a non-ether solvent/an aqueous alkali solution.

(2) The process for producing a cyanoethyl ether according to (1), wherein

the non-ether solvent is a substituted benzene.

(3) The process for producing a cyanoethyl ether according to (2), wherein

the substituted benzene is toluene.

(4) The process for producing a cyanoethyl ether according to any one of (1) to (3), wherein

the aqueous alkali solution is an aqueous solution of an alkali metal hydroxide or an alkaline earth metal hydroxide.
(5) The process for producing a cyanoethyl ether according to any one of (1) to (4), wherein

a compound represented by the following formula (III) is used in the reaction between the alcohol and the acrylonitrile:

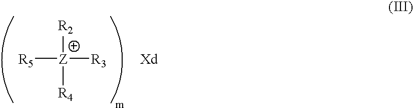

wherein

each of R₂, R₃, R₄, and R₅ independently represents an alkyl group or an aryl group;

Z represents a nitrogen atom or a phosphorus atom;

m represents an integer of from 1 to 3;

X represents a charge-balancing counterion; and

d represents a number necessary for balancing the charge.

(6) The process for producing a cyanoethyl ether according to any one of (1) to (5), wherein

the alcohol and the cyanoethyl ether are compounds represented by the following formulas (I) and (II), respectively:

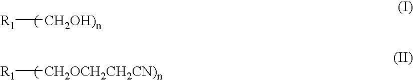

wherein

in the formula (I), R₁ represents an n-valent organic group and n represents an integer of 1 or greater; and

in the formula (II), R₁ and n have the same meanings as defined above, respectively.

### DETAILED DESCRIPTION OF THE INVENTION

The invention will hereinafter be described specifically.

The invention provides a production process of a cyanoethyl ether by at least reacting an alcohol and acrylonitrile in the presence of a two-phase system comprised of a non-ether organic solvent and an aqueous alkali solution which are incompatible with each other.

The non-ether solvent usable in the invention is not particularly limited and examples include cyclohexane, cyclohexanone, hexane, heptane, chlorobenzene, dichlorobenzene, trifluoromethylbenzene, toluene, and xylene. Of these, toluene and xylene are preferred, with toluene being more preferred.

The alkali of the aqueous alkali solution usable in the invention is not particularly limited and it is selected from, for example, alkali metal hydroxides and alkaline earth metal hydroxides. The alkali metal hydroxides are preferred. The alkali metal hydroxides are preferably sodium hydroxide and potassium hydroxide, with potassium hydroxide being more preferred. It is appropriate to use the alkali in an amount of from 5 to 200 mol %, preferably from 10 to 190 mol %, more preferably from 10 to 180 mol % relative to an alcohol.

In the invention, the reaction temperature is from 0 to 140° C., preferably from 5 to 100° C., more preferably from 5 to 30° C.

A content of the non-ether solvent relative to an alcohol is preferably from 1 to 100 V/W %, more preferably from 1 to 50 V/W %, still more preferably from 2 to 20 V/W %.

An acrylonitrile content is preferably from 0.95 to 20 equivalents, more preferably from 0.95 to 10 equivalents, still more preferably from 0.95 to 5 equivalents, each per equivalent of an alcohol.

The alcohol to be used in the invention will next be described.

The alcohol to be used in the invention is not particularly limited. Examples of it include compounds represented by the following formula (I):

R₁CH₂OH)ₙ (I)

In the formula (I), R₁ represents a linear or cyclic, n-valent organic group; n represents an integer of 1 or greater, preferably an integer from 2 to 10, more preferably an integer from 3 to 6. Specific examples of R₁ are shown below, but the invention is not limited by them.

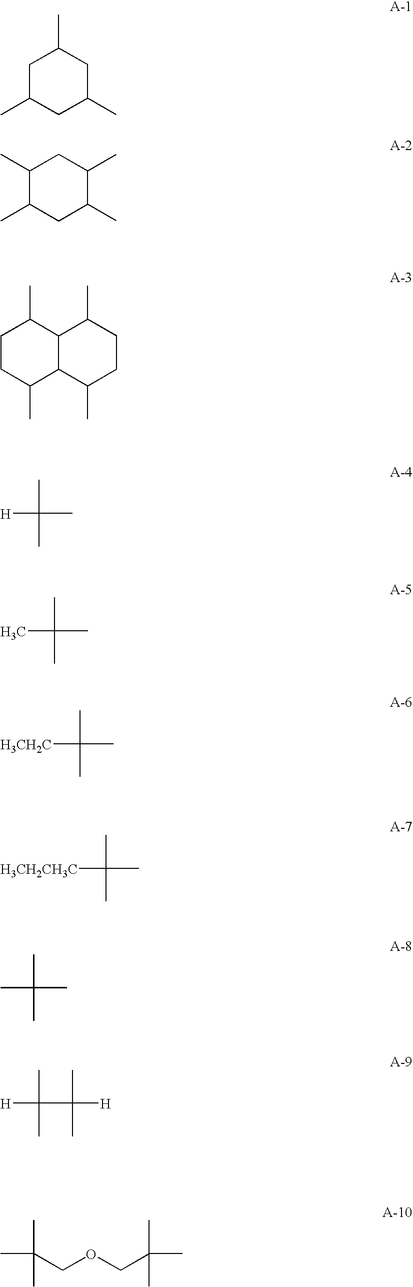

A reaction accelerator usable in the invention will next be described.

The reaction accelerator usable in the invention is not particularly limited. Examples of it include compounds represented by the following formula (III):

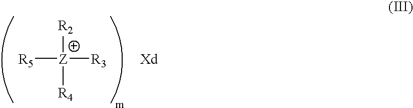

In the formula (III), each of R₂, R₃, R₄, and R₅ independently represents an alkyl group or an aryl group. Examples of the alkyl group include substituted or unsubstituted alkyl groups having not greater than 30 carbon atoms (which may be referred to as Cs or carbons), preferably not greater than 18 Cs. Specific examples include methyl, ethyl, propyl, butyl, pentyl, hexyl, octyl, dodecyl, hexadecyl, octadecyl, chloroethyl, bromopropyl, benzyl, and methoxymethyl. Examples of the aryl group include substituted or unsubstituted aryl groups having not greater than 30 Cs, preferably not greater than 18 Cs. Specific examples include phenyl, nitrophenyl, and cyanophenyl.

In the formula (III), Z represents a nitrogen atom or a phosphorus atom, preferably a nitrogen atom and m represents an integer of from 1 to 3.

X represents an anion as a charge-balancing counterion. The anion may be either an inorganic anion or an organic anion. Examples of it include halogen anion (such as fluorine ion, chlorine ion, bromine ion, and iodine ion), substituted arylsufonate ion (such as p-toluenesulfonate ion and p-chlorobenzenesulfonate ion), aryldisulfonate ion (such as 1,3-benzensulfonate ion, 1,5-naphthalenedisulfonate ion, and 2,6-naphthalenedisulfonate ion), alkylsulfate ion (such as methylsulfate ion), sulfate ion (such as monovalent monohydrogen sulfate ion and divalent sulfate ion), phosphate ion (such as monovalent dihydrogen phosphate ion, divalent monohydrogen phosphate ion, and trivalent phosphate ion), hydroxide ion, isocyanate ion, perchlorate ion, tetrafluoroboron ion, hexafluorophosphoris ion, picrate ion, acetate ion, and trifluoromethanesulfonate ion.

In the formula (III), d represents the number necessary for balancing the charge, preferably an integer from 1 to 3.

Specific examples of the compound represented by the formula (III) will hereinafter be shown. The invention is not limited by them.

The reaction accelerator is added in an amount of preferably from 0.1 to 100 mol %, more preferably from 0.1 to 50 mol %, still more preferably from 0.1 to 10 mol % relative to the alcohol.

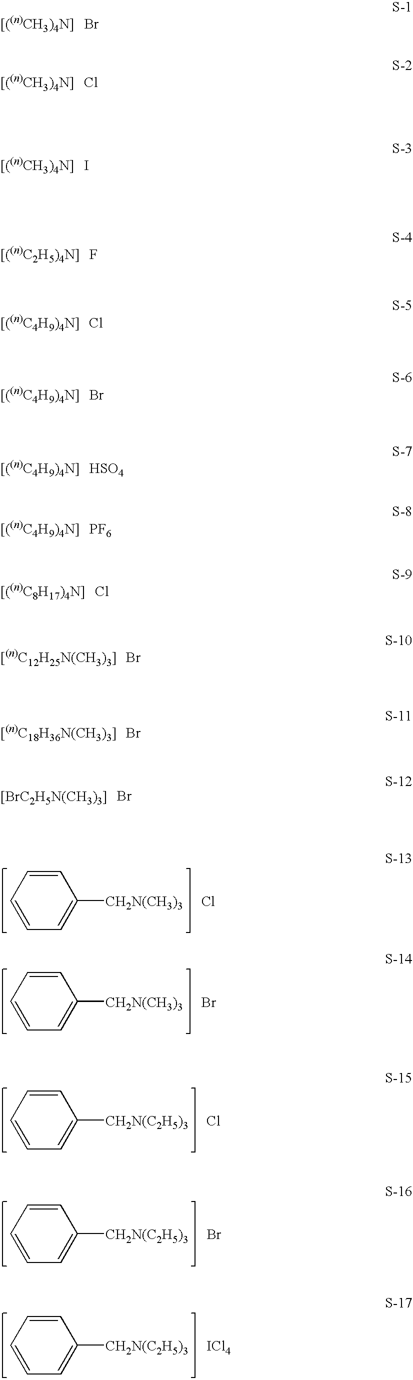

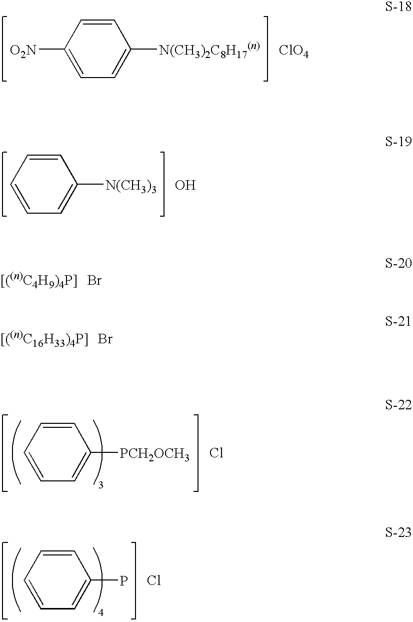

The cyanoethyl ether available by the process of the invention will hereinafter be described.

The cyanoethyl ether available by the invention is not particularly limited. Examples of it include compounds represented by the formula (II):

R₁CH₂OCH₂CH₂CN)ₙ (II)

In the formula (II), R₁ and n have the same meanings as described above in the formula (I). Specific examples of the compound represented by the formula (II) will next be described but the invention is not limited by them.

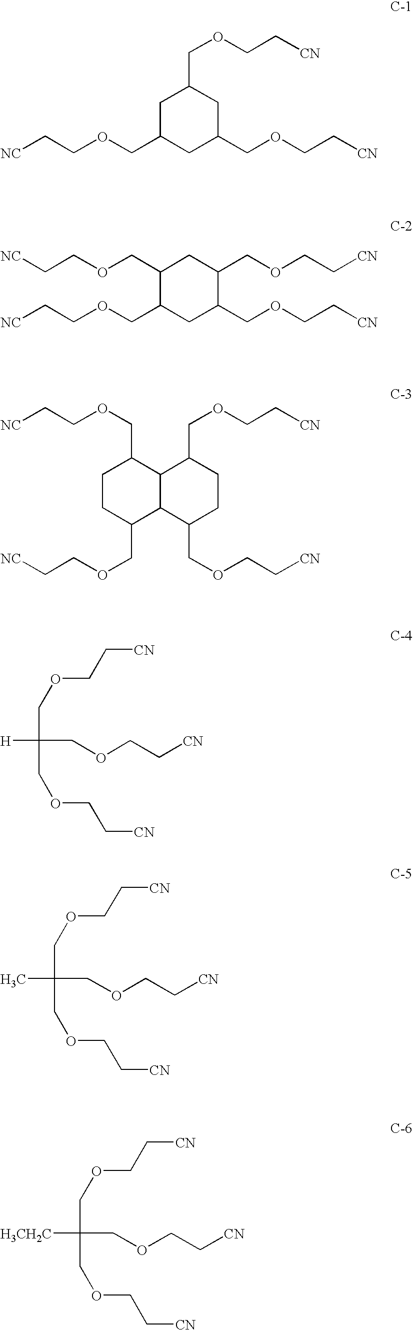

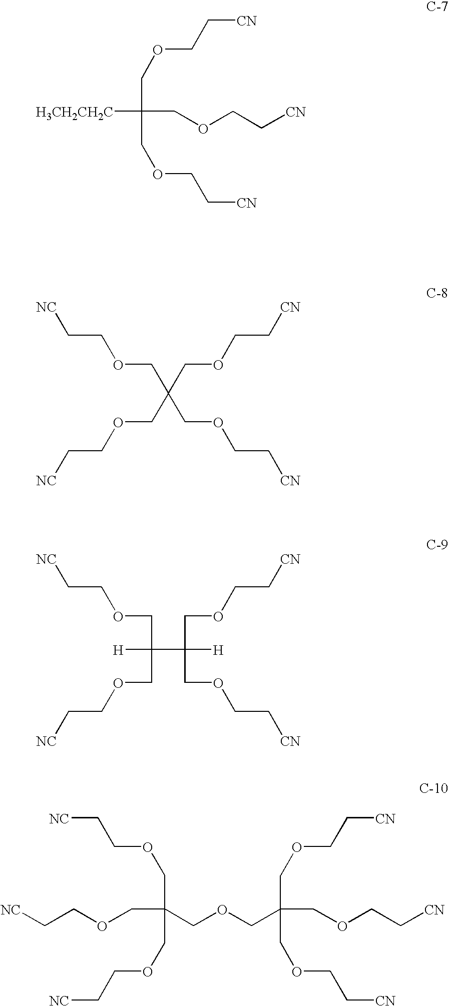

### EXAMPLES

The invention will hereinafter be described in further detail by examples. It should however be borne in mind that the invention is not limited to or by them.

### Example 1

### Synthesis of Exemplified Compound C-8

Pentaerythritol (PE) (150 g, 1.10 mol) and 120 ml (66 mol % relative to PE) of a 40 wt. % aqueous solution of potassium hydroxide are added to 525 ml of toluene. Acrylonitrile (953.4 g, 17.6 mol) is added dropwise to the resulting mixture at from 15 to 25° C. over 1 hour and a half. After stirring at from 20 to 25° C. for 3 hours, 975 ml of toluene and 1200 ml of 5 wt. % brine are added to the resulting pale yellow reaction mixture. The resulting mixture is stirred for several minutes to separate it into layers. The organic layer is washed twice with 1200 ml of 5 wt. % brine and then washed twice with 20 ml of concentrated hydrochloric acid and 1200 ml of 5 wt. % brine. Moreover, the organic layer is washed several times with 1200 ml of 5 wt. % brine and then dried over anhydrous magnesium sulfate. After filtration, the solvent is distilled off under reduced pressure to yield 419.7 g of a colorless oil. Bis(cyanoethyl)ether, that is, a by-product contained in the concentrated residue is distilled off under reduced pressure to yield 372 g (yield: 97%) of Exemplified Compound C-8 (purity by HPLC (Column: “SHISEIDO CAPCELL PAK”, trade name, eluent: methanol/water): 99%) in the form of a colorless oil. The colorless oil thus obtained becomes a white solid when left to stand at room temperature.

¹H-NMR (CDCl₃): δ 3.67 (t, J=6.0 Hz, 8H), 3.49 (S, 8H), 2.61 (t, J=6.0 Hz, 8H)

### Example 2

### Synthesis of Exemplified Compound C-8

Pentaerythritol (PE) (21.36 g, 0.156 mol) and 17.1 ml (66 mol % relative to PE) of a 40 wt. % aqueous solution of potassium hydroxide are added to 74.8 ml of xylene. Acrylonitrile (133.2 g, 2.51 mol) is added dropwise to the resulting mixture at from 15 to 25° C. over 1 hour. After stirring at from 20 to 25° C. for 3 hours, 138 ml of toluene and 170 ml of 5 wt. % brine are added to the resulting pale yellow reaction mixture. The resulting mixture is stirred for several minutes to separate it into layers. The organic layer is washed twice with 170 ml of 5 wt. % brine and then, washed twice with 4 ml of concentrated hydrochloric acid and 170 ml of 5 wt. % brine. Moreover, the organic layer is washed several times with 170 ml of 5 wt. % brine and then dried over anhydrous magnesium sulfate. After filtration, the solvent is distilled off under reduced pressure to yield 59.7 g of a colorless oil. Bis(cyanoethyl)ether, that is, a by-product contained in the concentrated residue is distilled off under reduced pressure to yield 50.4 g (yield: 92%) of Exemplified Compound C-8 (purity by HPLC: 99%) in the form of a colorless oil. The colorless oil thus obtained becomes a white solid when left to stand at room temperature.

### Example 3

### Synthesis of Exemplified Compound C-5

Trimethylolethane (TME) (60.08 g, 0.50 mol) and 8.4 ml (12 mol % relative to TME) of a 40 wt. % aqueous solution of potassium hydroxide are added to 210 ml of toluene. Acrylonitrile (159.2 g, 3.0 mol) is added dropwise to the resulting mixture at from 15 to 25° C. over 50 minutes. After stirring at from 20 to 25° C. for 2 hours, 390 ml of toluene and 750 ml of 5 wt. % brine are added to the resulting pale yellow reaction mixture. The resulting mixture is stirred for several minutes to separate it into layers. The organic layer is washed twice with 750 ml of 5 wt. % brine and then washed with 12 ml of concentrated hydrochloric acid and 750 ml of 5 wt. % brine. Moreover, the organic layer is washed several times with 750 ml of 5 wt. % brine and then dried over anhydrous magnesium sulfate. After filtration, the solvent is distilled off under reduced pressure to yield 159.1 g of a colorless oil. Bis(cyanoethyl) ether, that is, a by-product contained in the concentrated residue is distilled off under reduced pressure to yield 134 g (yield: 96%) of Exemplified Compound C-5 (purity by HPLC: 99%) in the form of a colorless oil.

¹H-NMR (CDCl₃): δ 3.65 (t, J=6.0 Hz, 6H), 3.38 (S, 6H), 2.60 (t, J=6.0 Hz, 6H), 1.60(S,3H).

### Example 4

### Synthesis of Exemplified Compound C-5

Trimethylolethane (TME) (60.08 g, 0.50 mol) and 6.4 ml (12 mol % relative to TME) of a 40 wt. % aqueous solution of potassium hydroxide are added to 210 ml of toluene. Acrylonitrile (159.2 g, 3.0 mol) is then added dropwise to the resulting mixture at from 15 to 25° C. over 50 minutes. After stirring at from 20 to 25° C. for 4 hours, 390 ml of toluene and 750 ml of 5 wt. % brine are added to the resulting pale yellow reaction mixture. The resulting mixture is stirred for several minutes to separate it into layers. The organic layer is washed twice with 750 ml of 5 wt. % brine and then washed with 12 ml of concentrated hydrochloric acid and 750 ml of 5 wt. % brine. Moreover, the organic layer is washed several times with 750 ml of 5 wt. % brine and then dried over anhydrous magnesium sulfate. After filtration, the solvent is distilled off under reduced pressure to yield 156 g of a colorless oil. Bis(cyanoethyl)ether, that is, a by-product contained in the concentrated residue is distilled off under reduced pressure to yield 132.6 g (yield: 95%) of Exemplified Compound C-5 (purity by HPLC: 98%) in the form of a colorless oil.

### Example 5

### Synthesis of Exemplified Compound C-8

Pentaerythritol (PE) (10 g, 0.073 mol), 8 ml (118 mol % relative to PE) of a 50 wt. % aqueous solution of potassium hydroxide, and 0.1 g (0.73 mol % relative to PE) of benzyltrimethylammonium chloride are added to 35 ml of toluene. After acrylonitrile (15.58 g, 0.293 mol) is added dropwise to the resulting mixture at from 15 to 20° C. over 3 hours, 45 ml of toluene is added to the resulting pale yellow reaction mixture to separate it into layers. The organic layer is washed twice with 72 ml of 5 wt. % brine, followed by washing twice with 1.5 ml of concentrated hydrochloric acid and 72 ml of 5 wt. % brine. Moreover, the organic layer is washed several times with 170 ml of 5 wt. % brine and then dried over anhydrous magnesium sulfate. After filtration, the solvent is distilled off under reduced pressure to yield 25 g (yield: 96%) of Exemplified Compound C-8 (purity by HPLC: 98%) in the form of a colorless oil. The colorless oil thus obtained becomes a white solid when left to stand at room temperature.

### Comparative Example 1

### Synthesis of Exemplified Compound C-8

Pentaerythritol (PE) (6.29 g, 0.046 mol) and 5 ml (66 mol % relative to PE) of a 40 wt. % aqueous solution of potassium hydroxide are added to 22 ml of dioxane. Acrylonitrile (39.2 g, 0.738 mol) is added dropwise to the resulting mixture at from 15 to 25° C. over 50 minutes. After stirring at from 20 to 25° C. for 3 hours, 100 ml of toluene and 80 ml of 5 wt. % brine are added to the resulting yellow reaction mixture. The resulting mixture is stirred for several minutes to precipitate a yellow solid. The yellow solid is filtered out, followed by separation into layers. The organic layer is washed twice with 100 ml of 5 wt. % brine and then washed twice with 1.2 ml of concentrated hydrochloric acid and 100 ml of 5 wt. % brine. Moreover, the organic layer is washed several times with 100 ml of 5 wt. % brine and then dried over anhydrous magnesium sulfate. After filtration, the solvent is distilled off under reduced pressure to yield 14.2 g of a colorless oil. Bis(cyanoethyl)ether, that is, a by-product contained in the concentrated residue is distilled off under reduced pressure to yield 14.2 g (yield: 88.2%) of Exemplified Compound C-8 (purity by HPLC: 95%) in the form of a colorless oil. The colorless oil thus obtained becomes a white solid when left to stand at room temperature.

### Comparative Example 2

### Synthesis of Exemplified Compound C-8

(performed with reference to Tetrahedron, 55(10), 2941-2956(1999))

Pentaerythritol (PE) (6.84 g, 0.05 mol), 2 ml of water, and 1 ml (12.1 mol % relative to PE) of a 40 wt. % aqueous solution of potassium hydroxide are added to 20 ml of dioxane. Acrylonitrile (16.2 g, 0.305 mol) is added dropwise to the resulting mixture at 0° C. over 10 minutes. After stirring at room temperature for 48 hours, the solvent is distilled off under reduced pressure to obtain a yellowish brown oil. Washing of the resulting yellowish brown oil with 100 ml of dichloromethane and 50 ml of 10 wt. % brine precipitates a yellowish brown solid. The resulting yellowish brown solid is filtered out, followed by separation into layers. After the water layer is extracted twice with 25 ml of dichloromethane, the resulting extract is mixed with the first organic layer. The resulting mixture is dried over anhydrous magnesium sulfate. The solvent is distilled off under reduced pressure to yield 15.9 g of a colorless oil. Bis(cyanoethyl)ether, that is, a by-product contained in the concentrated residue is distilled off under reduced pressure to yield 14.4 g (yield: 82.3%) of Exemplified Compound C-8 (purity by HPLC: 96%) in the form of a colorless oil. The colorless oil thus obtained becomes a white solid when left to stand at room temperature.

According to the invention, a high-purity cyanoethyl ether useful as medicaments, agricultural chemicals and various functional materials, or intermediates thereof can be produced in a high production yield by an industrially advantageous process while ensuring safety in human health and environment.

The entire disclosure of each and every foreign patent application from which the benefit of foreign priority has been claimed in the present application is incorporated herein by reference, as if fully set forth.

What is claimed is:

1. A process for producing a cyanoethyl ether, comprising:
reacting an alcohol and acrylonitrile in a two-phase system of a non-ether solvent/an aqueous alkali solution.

2. The process for producing a cyanoethyl ether according to claim 1, wherein the non-ether solvent is a substituted benzene.

3. The process for producing a cyanoethyl ether according to claim 2, wherein the substituted benzene is toluene.

4. The process for producing a cyanoethyl ether according to claim 1, wherein
the aqueous alkali solution is an aqueous solution of an alkali metal hydroxide or an alkaline earth metal hydroxide.

5. The process for producing a cyanoethyl ether according to claim 1, wherein
a compound represented by the following formula (III) is used in the reaction between the alcohol and the acrylonitrile: 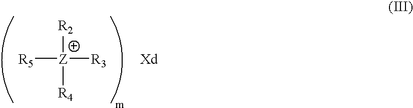
wherein
each of R₂, R₃, R₄, and R₅ independently represents an alkyl group or an aryl group;
Z represents a nitrogen atom or a phosphorus atom;
m represents an integer of from 1 to 3;
X represents a charge-balancing counterion; and
d represents a number necessary for balancing the charge.

6. The process for producing a cyanoethyl ether according to claim 1, wherein
the alcohol and the cyanoethyl ether are compounds represented by the following formulas (I) and (II), respectively: 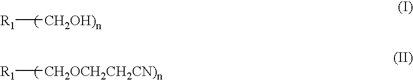
wherein
in the formula (I), R₁ represents an n-valent organic group and n represents an integer of 1 or greater; and
in the formula (II), R₁ and n have the same meanings as defined above, respectively.
